(19) United States
(12) Patent Application Publication
(10) Pub. No.: US 2010/0069897 A1
(43) Pub. Date: Mar. 18, 2010

(54) METHOD AND DEVICE FOR GENERATING A LASER BEAM, A LASER TREATMENT DEVICE AND A LASER DETECTION DEVICE

(75) Inventors: Bart Willem Jan Spikker, Eindhoven (NL); Rieko Verhagen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/532,461

(22) PCT Filed: Mar. 26, 2008
(86) PCT No.: PCT/IB08/51130

§ 371 Date: Sep. 22, 2009

(30) Foreign Application Priority Data
Mar. 29, 2007 (EP) 07105177.5

(51) Int. Cl. A61B 18/20; H01S 3/11; H01S 3/101

(52) U.S. Cl. 606/9; 372/10; 372/75

(57) ABSTRACT

The invention relates to a laser device, comprising a laser material (1) brought into a simmer mode. A controllable source (7, 17) of additional energy (16, 20) supplies energy to the laser material (1), such that in only a desired part of the laser material (1) a lasing threshold is exceeded, and a laser beam (10) is emitted from only a desired part of the laser surface. This device makes possible to provide a laser beam in just the desired part of the laser, which allows a flexible and localized output. The invention further relates to a hair-removing device comprising a laser device according to the invention and further comprising an optical system (6) for focusing the laser beam pulses on a focal spot (12) and for positioning the focal spot in a target position, wherein the optical system (6) comprises a movable lens or a plurality of individually addressable lenses.

### FIELD OF THE INVENTION

The invention relates to a method and device for generating laser pulses.

The invention also relates to a hair-removing device using such method and/or device.

### BACKGROUND OF THE INVENTION

Methods of generating laser pulses are well-known. Very generally, they consist of bringing about a population inversion in a suitable material, such that stimulated emission may occur, and further increasing the intensity by reflecting the generated radiation between mirrors in a laser cavity. Such lasers may also be pumped and pulsed lasers, which are able to generate very short and very intense pulses of radiation.

A disadvantage of lasers and the way of generating a laser beam as mentioned above is that it is rather inflexible as regards the beam. In fact, it is there or not, with at most a variable power, repetition frequency and/or frequency.

### OBJECT OF THE INVENTION

It is an object of the present invention to provide a method and device for generating a laser beam with increased flexibility, in particular as regards the beam properties.

### SUMMARY OF THE INVENTION

The above object of the invention is achieved, in a first aspect, with a method of generating a laser beam, comprising providing a laser device, having a laser material, with a surface from which a laser beam is emittable, and a Q-switch in a laser cavity between cavity mirrors, bringing the laser material into a simmer mode by supplying energy to the laser material, providing additional laser material absorbable energy to the laser material, such that in only a desired part of the laser material a lasing threshold is exceeded, and a laser beam is emitted from only a desired part of said surface. The invention allows the generation of a laser beam in only a desired part of the laser material. This increases the flexibility since it allows the generation of a beam in various desired positions. This in turn allows various subsequent effects, if such effects are made position-dependent. The simplest example is of course where the energy of the laser beam is to be emitted or supplied position-dependently, in which case prior art devices would require an additional and movable part in the form of e.g. a positioning mirror or the like. Further examples of use of the present invention will be given below. Another advantage is that only part of the laser material, i.e. a part less than the full laser material, is used to generate the laser pulse. Furthermore, since the laser beam generation is distributed, the average time between two pulses for the same position within the laser material is increased, compared to lasing across the entire laser material. Hence, the pumping intensity may be lower, and temperature control in the material is made easier and the service life of the laser material may be increased.

A laser material, often but not necessarily a laser crystal, may be excited by supplying energy, e.g. energy that is absorbable by the laser material, preferably substantially continuously. Generally, it will then fluoresce back to the ground state. If more energy is supplied, there will be more and more excitation (and fluorescence), until a simmer threshold is reached, at which amplified spontaneous emission will occur, although not yet a laser output, since the Q-switch still absorbs too much of the radiation incident thereon. If, subsequently, a part of the volume is provided with sufficient energy, the following phenomena will occur. First, the additional energy is absorbed by the laser material, and increased spontaneous emission is achieved, because the inversion level is raised above the laser threshold. Then, the generated intensity is such that, at least in the part of the Q-switch corresponding to that part of the laser material, will switch from a high-absorption state to a low absorption state. This enables the laser function to occur, since the generated radiation will now be reflected back and forth at ever increasing intensity, releasing the energy that was stored in a simmer mode. The result will be a short but high-power laser pulse. Thereafter, pumping will again bring the relevant part of the laser material to a simmer state, and the process has come full cycle. The passive Q-switch as described above is in particular a saturable absorber Q-switch.

Preferably, the Q-switch is positioned opposite the surface of the laser material from which the laser beam is to be emitted. This allows the use of the radiation emitted in a simmer mode.

In the above, “corresponding” relates to the part of the laser material that is in the path of the ultimately emitted laser beam, which supplies a part of the laser beam energy.

Note that at the start, amplified spontaneous emission occurs in all directions, but the Q-switch, or the side faces of the laser material, stops most of that radiation. Only if a switching threshold is exceeded, lasing may start. This will be the case for the parts of the Q-switch closest to the additionally excited volume. This automatically limits the beam width. A general remark here is that the supplied additional energy need not have a laser wavelength, and so does not interfere with the laser process itself.

One may think of the above of the generation of a laser within the laser material, since only part thereof is brought into the laser mode.

In embodiments, additional laser material absorbable energy is supplied to the laser material, such that in a plurality of desired parts of the laser material a lasing threshold is exceeded, and a laser beam is emitted from said plurality of desired parts of said surface. Herein, the plurality of parts preferably have a total cross-sectional area that is smaller than the surface of the laser device that can emit a laser beam, without such embodiment being excluded, but the latter merely comes down to full laser action for the total laser material.

In particular, the step of bringing the laser material into simmer mode is controlled by controlling an input power of the supplied energy. This prevents the laser material from going accidentally into lasing mode, or from falling out of simmer mode. This control step may be brought about by measuring the radiation emitted in simmer mode. This may be made to lie between values corresponding to limiting values at simmer threshold and laser threshold, respectively.

The step of bringing the laser material into simmer mode may be performed with the use of a pump means, in particular an optical pump, which may comprise one or more light sources, in particular a (laser) diode or pump laser, emitting radiation of a suitable wavelength and power. Pumping refers to exciting of the atoms, molecules etc. in order to achieve some level of population inversion, and is per se sufficiently known in the art. Pumping could be performed in other ways than supplying suitable optical energy, such as by supplying an electrical current or by chemical reactions.

The step of supplying the additional energy may comprise providing a beam, preferably one or more pulses, of radiation that is focused in the desired part of the laser material. This may be done from, in principle, anywhere outside the laser material, since the focusing controls the desired position, but is preferably provided from a side surface of the laser material, with respect to the laser beam to be produced. Such provision of energy may be performed with a pulsed light source such as a pulsed diode, a flash lamp et cetera, in combination with a focusing element, such as a lens. Aiming the focus may be brought about by an addressable mirror and so on.

Alternatively or additionally, it is possible to direct at least one pulse of additional energy to the laser material, from a side surface with respect to, or opposite the surface of the laser material from which the laser beam is to be emitted. This additional energy need not be focused, which is advantageous because it requires less optics etcetera. Examples are again a pulsed diode, a pulsed laser and so on. Here, the pulse is amplified many times by the laser material, so the pulse can be weak, in fact need only be strong enough to increase the inversion level from the simmer level to beyond the laser threshold. The additional energy is preferably, but not necessarily, supplied in a substantially parallel beam that crosses the desired cross-sectional part of the laser material. The intended laser beam output face of the laser material is easily determined, and is often the side at which the Q-switch is positioned. This often comprises the side opposite the high-reflectance side of the laser cavity. In other words, the additional energy is now input at the side of the high-reflectance mirror. But this high reflectance holds for the laser wavelength, and not necessarily for the additional energy.

To position the additional energy, i.e. to select the desired output position, or plurality of positions, of the generated laser pulse(s) as output by the laser material, the source of additional energy is controllable, in other words the additional energy is provided controllably. For example, the source comprises a plurality of individually controllable subsources, such as a matrix of diodes. Alternatively, the source could comprise a source with a beam positioning device such as a pivotable mirror, and optionally a lens, or a focusing mirror, to position the additional energy in the laser material.

The invention also relates to an optical detection method for detecting objects in a medium, in particular hair in skin, the method comprising providing optical simmer radiation by means of a laser material that is brought into simmer mode, preferably with a method according to the above aspect of the invention, and detecting returning radiation with an optical detector, wherein said returning radiation is guided through and amplified by the laser material in simmer mode. Herein, good use is made of the simmer radiation, and in particular of the fact that the returning radiation will be amplified in the laser material. Other background radiation, from external sources, having other wavelengths, will not be amplified. Now, use may be made of a simple silicon photodiode, which is less complex and is cheaper than e.g. avalanche photodiodes, although these too may be used.

Preferably, the step of guiding the radiation through the laser material comprises the step of redirecting the radiation to go through the laser material more than once, more preferably by means of a mirror, prism or beam splitter. This additionally enhances the returning radiation, and more than the background radiation. The beam splitter may further be used to guide selected radiation towards the detector.

In embodiments, the method comprises the step of polarizing the optical simmer radiation. Particularly the detection step comprises rejecting radiation that is cross-polarized with respect to the returning radiation. Even more particularly, the polarization is circular polarization, or at least elliptical polarization with an axial ratio of between 1:1 and 1:2. Use of polarized light allows to discern between radiation reflected at the skin surface and radiation that is e.g. scattered at a subcutaneous hair. Furthermore, circularly, or not too extremely elliptically, polarized light is even more useful in optical detection, as was described in EP 06125915.6.

Advantageous methods further comprise detecting at least one hair position, and providing additional energy in at least one position in the laser material, such that at least one laser beam is emitted into the at least one hair position. This is a very expedient combination of the above described detection method which uses laser material in simmer mode, and the method of the first aspect of the invention which also uses a laser material in simmer mode, by combining their advantages.

The invention in particular relates to a laser device, comprising a laser cavity with cavity mirrors, and with a laser material having a surface from which a laser beam is emittable and a Q-switch positioned in the laser cavity, a pump means, able and arranged to supply energy to the laser material to bring the laser material into a simmer mode, and a controllable source of additional energy, arranged to supply laser material absorbable energy to the laser material, such that in only a desired part of the laser material a lasing threshold is exceeded, and a laser beam is emitted from only a desired part of said surface. Such a device is in particular suitable for performing a method according to the invention, and provides at least the advantages already mentioned above. Therefore, advantageous embodiments will be discussed only briefly, where possible, referring the reader to the corresponding parts of the description of the method above.

Herein, a “controllable light source” is a light source that is arranged to emit light, i.e. not necessarily visible optical radiation, in a controlled part of the laser material. This could be fixed and only on-off, or could be variable as regards position.

In particular, the Q-switch may be a passive Q-switch, such as a saturable absorber, able to go from a highly absorptive state to a highly transmissive state. The Q-switch is preferably positioned at a side of the laser material that faces away from the intended laser beam output direction. In this way, the simmer radiation may be used for desired purposes, such as illuminating an object to be treated with the laser output.

The pump means may be of any suitable type, such as an electrical pump, adding energy through electrical fields or the like. Advantageously, the pump means comprises an optical pump, since this allows easy and versatile control over the pumping. The optical pump may comprise one or more diodes, such as laser diodes, a pumping laser which may have relatively low power, and so on. The wavelength and power may be such that the laser material is pumped to a suitable population distribution, above simmer threshold but below laser threshold.

Similarly, the source of additional energy may comprise one or more diodes, such as laser diodes, a pulsed laser, with relatively low pulse energy, a flash lamp, and so on. The source of additional energy is preferably arranged to supply the energy in a substantially parallel beam or in a focused beam.

The source of additional energy may be positioned at the sides of the laser material, i.e. at the sides not crossing the path of the laser beam. In that case, the source may either supply the additional energy in a beam smaller than the cross-sectional area of the laser material, or preferably in a focused beam. The source may also supply the additional energy through a front surface or, advantageously, a back surface of the laser material, i.e. a surface that is substantially perpendicular to the laser radiation output by the laser material.

The source of additional energy may be controllable in that it comprises a plurality of independently addressable radiation sources, such as a matrix of (laser) diodes, or one or more radiation sources with separate shutters or the like. It is also possible that there is provided a radiation source and a controllable beam guiding device, such as a controllable mirror or set of mirrors, and/or one or more lenses.

The applications of methods and devices according to the invention as described above can be numerous.

As a first example, flexible and fast addressing of laser pulse devices is possible. For instance, if a bundle of optical fibers is provided, it becomes possible to send laser pulses through one or more selected fibers of the bundle by correspondingly addressing one or more associated sources of additional radiation. This may prove valuable in transmitting information or performing calculations in an optical computer.

A second example will be elaborated below, in connection with another aspect of the present invention.

This aspect relates to a laser-based hair-removing device, comprising a laser source arranged to provide laser beam pulses, an optical system for focusing the laser beam pulses on a focal spot and for positioning the focal spot in a target position, wherein the laser source comprises a laser device according to the invention. This device may use the localized beam generation in order to control the supplying of laser pulses to desired locations, in this case in particular hairs.

Preferably, the optical system comprises a movable lens, or a plurality of individually addressable lenses which are not necessarily movable. This adds flexibility to the positioning.

In particular, the laser source is arranged to provide the laser beam pulses with a predetermined pulse time, and a dimension of the focal spot and a power of the generated laser beam pulse are such that, in the focal spot, the laser beam pulse has a power density that is above laser induced optical breakdown (LIOB) threshold for hair tissue for that predetermined pulse time.

This device offers the advantage, over known devices such as that disclosed in WO2005/011510, that it may be kept more compact and more efficient. Especially the optical system may be kept much smaller, since for LIOB, a combination of a very small focus and a high NA is required. This necessitates a large lens to ensure the required optical quality. On the one hand, devices with movable mirrors as the beam manipulator after the optical system (sometimes called post-objective scanning) suffer from the mirror interfering with the optical system to focus the laser beam pulse, due to its desirable high numerical aperture (NA) and the associated rapidly converging beam. On the other hand, other devices with a configuration in which a manipulator mirror is positioned before the optical system (or pre-objective scanning) need a very large diameter lens, due to the field of view being very limited at such high numerical aperture. For example, for an area to be scanned (i.e. treated) of about 20 mm, the lens would need to be about 200 mm in diameter for an exemplary suitable NA. The device according to the present invention overcomes this problem by distributing the laser beam pulse over a desired scan area by means of the laser device described in the first aspect of the invention, and by providing a movable lens or a plurality of individually addressable lenses.

The movable lens may be moved across the scan area. In a suitable position for the laser beam to be focused on the target position, the laser beam pulse is provided. Because that laser beam passes only through a controllable part of the surface area of the laser material, it is easy to limit the beam to go only through the movable lens, and thus to prevent a part of the beam from circumventing the lens.

In the case of the plurality of individually addressable lenses, the desired lens or lenses are addressed, i.e. supplied with a laser beam (pulse). In this case, the lenses need not be movable, although additional flexibility is achieved when they are.

A special advantage of the devices and methods according to the invention relates to the use of laser material in simmer mode. Because the laser material in simmer mode already has a high population inversion, the additionally needed energy for the laser threshold to be reached is very small, and can be given in a well-defined and short pulse. This allows a tight and accurate control over the timing of the laser, and hence also over the precise position of the laser beam pulse as emitted through a movable lens. In other words, the lens need not be stopped in order to have a sufficiently accurate position control. This is in contrast to a laser material that is brought from ground state to laser mode with the trigger pulse only, because then much more time is needed, as is visible in FIG. 2. The associated uncertainty about the actual start of the laser pulse decreases this temporal, and thus spatial, accuracy.

Preferably, a control unit is provided that controls the laser device and the optical system, such that the generation of the desired laser beam pulse, through supply of additional energy, and the positioning of the focal spot are coupled. In other words, the control unit is arranged to ensure that a laser beam pulse is generated at a suitable position in the laser material, and that the optical system is arranged to position the resulting focal spot in the desired target position. In case a plurality of fixed lenses is used, this positioning is not an active movement, although the position for supplying additional energy of course also has an influence on the final focal spot position. A known relation between positions in the laser material and a position of the one or more lenses in the optical system is useful in this control, although experimental trial and error could also lead to satisfactory results.

In general, LIOB occurs in media, when the power density of the laser beam in the focal spot exceeds a threshold value which is characteristic of the particular medium. Below the threshold value, the particular medium has relatively low linear absorption properties for the particular wavelength of the laser beam. Above the threshold value, the medium has strongly non-linear absorption properties for the wavelength, which are the result of ionization of the medium and the formation of plasma, e.g. due to multi-photon absorption. The LIOB phenomenon results in a number of mechanical effects, such as cavitation and the generation of shock waves, which damage the medium (hair tissue) in positions surrounding the position of the LIOB phenomenon. The LIOB phenomenon has also been mentioned in document WO2005/011510, which relates to a shaving device.

It is noted that LIOB, and thus use of the device according to the present invention, is not dependent on type of skin or type of hair. LIOB relies on non-linear absorption, and is independent of type of skin and type of hair. In fact, the present device may be used for fair skin as well as dark skin, dark hairs and even colourless vellus hairs.

Hair tissue and skin tissue are transparent or semi-transparent for wavelengths between approximately 500 nm and 2000 nm. The linear absorption of the tissues, as well as scattering are low enough for LIOB to be possible. Particularly the wavelength of the laser beam is between 800 nm and 1400 nm, more particularly between 1000 and 1100 nm. For wavelengths within this range, the linear absorption properties and scattering properties are at a minimum, so that a maximum portion of the energy of the generated laser beam is used to cause the LIOB phenomenon in the focal spot of the laser beam, while in surrounding tissue only very small portions of the (scattered) energy is absorbed.

Further details relating to LIOB may be found in the introductory part of the above-mentioned document. Suffice it here to state that the threshold value of the power density is dependent on the pulse time. For example, for a pulse time of 10 ns, the threshold value is about 8*10¹¹ W/cm², although some references in the literature disclose lower values in this case, such as about 2*10¹⁰ W/cm². Such values may be readily obtained with even a low energy laser. Note that shorter pulse times, such as in the order of pico seconds or femto seconds are also effective, contrary to the minimum pulse time of 1 ms as required in WO00/62700. Note also that the laser need not have a fixed or predetermined pulse time. A variable pulse time is also possible.

It is noted that the required power density is defined in terms of the result to be achieved, in particular the laser beam and focusing system should generate a power density in the focal spot that is above the LIOB threshold value. However, with knowledge of said threshold value, which is obtained either by theory or by experiments, the skilled person will readily select the laser power and the optical system in order to achieve the required power density

It is remarked here that the present invention is advantageous when a high numerical aperture optical system is used, since there the gain in compactness is very great. Using a high numerical aperture lens in a laser hair removal device that uses LIOB is advantageous since it is safer for the skin surrounding the target area. A high numerical aperture ensures quick convergence and divergence of the laser beam outside the focal spot, such that outside that focal spot, there is reduced risk of tissue being damaged. In a particular embodiment, the optical system has a numerical aperture of at least 0.2, preferably at least 0.4, more preferably at least 0.6. Such values for the numerical aperture relate to safety for the overlying skin layers, in particular the epidermis. Since in particular the epidermis contains many chromophores such as melanin, the residual linear absorption in the epidermis is not negligible. Hence it is advantageous to keep the fluence, or energy density, in such layers sufficiently low. This may be achieved by providing a strongly focused laser beam, i.e. with a large angle of convergence, hence with large numerical aperture of the optical system. The laser beam then covers a sufficiently large area to keep the fluence in the epidermis within an acceptable range.

In the above, the movable lens, or the plurality of individually addressable lenses, which possibility is considered included in each of the features mentioned below, has a double function, both as a focusing element and as a beam manipulator for positioning the focal spot. It is however also possible to split these functions by providing a movable lens for focusing and another lens or a mirror system or the like for manipulating. In such embodiments, the optical system comprises a focusing system and a beam manipulating system. Embodiments hereof will be given below.

In a special embodiment, the device comprises a plurality of movable lenses, preferably arranged as a lens array. This allows a quicker scan, and also to provide more than one laser beam pulse at substantially the same time.

In an embodiment, the device comprises a lens mover, in particular an array mover, that is arranged to impart an oscillating, reciprocating or vibrating movement to the movable lens, in particular to the lens array. A useful array could be a linear array, a staggered array or the like. A staggered array, comprising e.g. a plurality of mutually shifted rows of lenses, has an advantage that a row of lenses covers inter-lens spaces of other rows, such that no spot need be missed when scanning or the like takes place. The lens (array) mover may be any mechanical mover means or actuator, suitable types being e.g. electromechanical and piezoelectrical actuators.

In another embodiment, the device comprises a lens mover or array mover which is arranged to impart a rotating movement or the movable lens or lens array, respectively. A useful array could be a circular array, preferably with a lens-free center, such as a hole.

In embodiments, the laser material, or at least the part of the laser device that is able to emit a laser beam, should have a size, or cross-sectional area, at least equal to the scanning area. In other words, e.g. in the example of the vibrating array, said size should be at least the size of the lens array plus twice the amplitude of the vibrating movement. Similar considerations hold for other lens array embodiments. The shape of the laser material, e.g. the laser crystal, is adapted similarly, such as a rectangular or circular rod.

The invention also relates, in another aspect, to an optical detection device, particularly to image subcutaneous parts of human skin, more particularly hairs. Such devices are known. A problem with such devices is that the signal returning from below the skin, in particular from subcutaneous hair parts or the like, is relatively weak, and especially the ratio between radiation incident on the skin, or reflected by its surface, on the one hand, and returning from the subcutaneous hair parts on the other hand, is very high. In order to reduce this ratio, it has been proposed to use a pinhole to “filter” the subcutaneous signal from the surface reflections and other background radiation, but this also further reduces the absolute subcutaneous signal. Still, complex and expensive detectors, such as an avalanche photodiode, are required for sufficient sensitivity. Note that simply increasing the intensity of the incident radiation to obtain a sufficient signal strength is often not allowed, e.g. when irradiating skin, owing to safety regulations.

It is an object of the present invention to provide an optical detection device with improved sensitivity and/or selectivity.

The above object is achieved with an optical detection device, particularly to image subcutaneous parts of human skin, more particularly hairs, the device comprising a laser cavity with cavity mirrors, and with a laser material having a surface from which radiation is to be emitted and a Q-switch positioned in the laser cavity, a pump means, able and arranged to supply energy to the laser material to bring the laser material into simmer mode, and a photodetector, arranged to detect emitted radiation that returns from an object to be detected. By using a laser material that is in simmer mode, a continuous-wave optical output is achieved which serves as illumination of the skin. Radiation that returns, inter alia from subcutaneous hair parts or the like, may be detected to form an image.

Advantageously, the photodetector is arranged and positioned to detect returning radiation that has passed through the laser material. In other words, the photodetector has such a position that it will detect returning radiation after this radiation has gone through the laser material, which is in simmer mode during use. The returning radiation is amplified in the laser material, which gives amplification of the returning emitted radiation only, not of the other background radiation, which often has a different wavelength. A suitable position of the detector could be opposite a light emission side of the laser material, which is a very simple solution. It is also possible to position the photodetector differently, through the use of some radiation guiding means.

Because of the amplification, the optical detector may be kept simple, and preferably comprises a photodiode, such as a simple silicon photodiode, although other types such as photomultiplier tubes or avalanche photodiodes are not excluded. This aspect may be combined with all advantageous features as described in connection with the other aspects of the invention. For example, the pump means may be an optical pump, such as one or more (laser) diodes and so on.

Note that the laser devices according to the invention may also comprise an optical detection device according to the invention, the latter thus having both inventive value as a component and as a stand alone device, without being used in combination with a laser-based hair removal device or even a general laser device. Also, particular embodiments of any laser device and any optical detection device according to the invention may be combined as desired. In such a combined case, it will be advantageous, to reduce complexity, for the laser cavities and laser materials of the devices to be combined. In other words, if a laser device of the invention comprises an optical detection device of the invention, the laser material and the laser cavity and so on are advantageously shared by both devices, although this is not necessary.

Advantageously, the optical detector comprises a pinhole that serves to select desired radiation and filter out unwanted background radiation, by limiting the field of view, and/or an imaging lens that images the returning radiation onto the photodetector. The absolute signal strength is now higher, which improves the signal-to-noise ratio, and signal detection in general. Note that the use of the detection device is not limited to detecting subcutaneous parts of hair in skin, but could be applied in other situations where a signal is to be determined from e.g. scattering, and in particular at objects within another medium.

Advantageously, the optical detection device comprises a mirror device, arranged such that it is able to redirect returning radiation through the laser material along a different, preferably parallel, path. This further improves signal strength. The position of the detector, pinhole et cetera may be adapted accordingly.

The optical detection device comprises, in an embodiment, a mirror device to guide the reflected radiation to the photodetector, in particular more than once through the laser material. The mirror device could comprise e.g. one or more plane mirrors. However, in an advantageous embodiment, the mirror device comprises at least one polarizing beam splitter. This allows to discern two types of polarization in the returning radiation. A big advantage is that reflection at the skin surface has a different effect on polarization of the radiation than has scattering, or other returning mechanisms, at subcutaneous hair parts. Hence, by providing such a polarizing beam splitter, one is able to further improve the ratio between desired radiation, i.e. returning from subcutaneous hair parts, and unwanted radiation, such as reflected at the skin surface.

In an embodiment, the optical detection device further comprises a Faraday rotator and a quarter lambda plate between the laser material and the focusing lens. The use of a quarter lambda plate allows the generation of circularly polarized light. Thereto, a linear polarizer may be included, if the emitted laser radiation is not (fully) linearly polarized. The advantages of the use of circularly polarized light have e.g. been described in co-pending European Patent Application EP 06125915.6.

In particular, the optical detection device comprises a hair recognition device, such as a CPU that processes the optical signals from the detector, and that is programmed suitably. Such programming (software) per se is known in the art.

In further advantageous embodiments, the optical detection device is part of a laser device according to the present invention. Then, the laser device, in particular the device for treating hairs, can advantageously use a detected signal from the present detection device in order to determine where to provide a laser pulse. All special features mentioned in the first aspect of the invention may also be part of such embodiments. As one example, without the additional features being limited thereto, there may be mentioned the provision of a laser cavity with mirrors and a Q-switch between the laser material and a mirror, preferably at the mirror facing away from the side of the intended laser beam. This allows to use the continuous emission in simmer mode, while the Q-switch allows the generation of a laser beam when switched to a transparent state, in particular by increased radiation incident thereon, such as from a start pulse. Herein, the device becomes a detection and “shooting” device in one.

In an expedient embodiment, the detection device comprises a shutter that is positionable in front of the detector. This shutter is advantageously controllable by a control device, to shut off the optical path to the detector when the device provides a laser pulse. In such case, scattered radiation of increased sensitivity might otherwise damage the detector.

### BRIEF DESCRIPTION OF THE DRAWINGS

These and other aspects of the invention are apparent from and will be elucidated with reference to the embodiments described hereinafter.

In the drawings:

FIG. 1 very generally depicts a device that shows various inventive features;

FIG. 2 diagrammatically shows a number of steps in the method of the invention;

FIG. 3 diagrammatically shows a cross-sectional view of a device according to the invention;

FIG. 4 diagrammatically shows a cross-sectional view of another device according to the invention;

FIG. 5 diagrammatically shows a detection device according to the invention; and

FIG. 6 diagrammatically shows a cross-sectional view of an alternative embodiment of the detection device shown in FIG. 5.

### DETAILED DESCRIPTION OF THE PREFERRED EMBODIMENTS

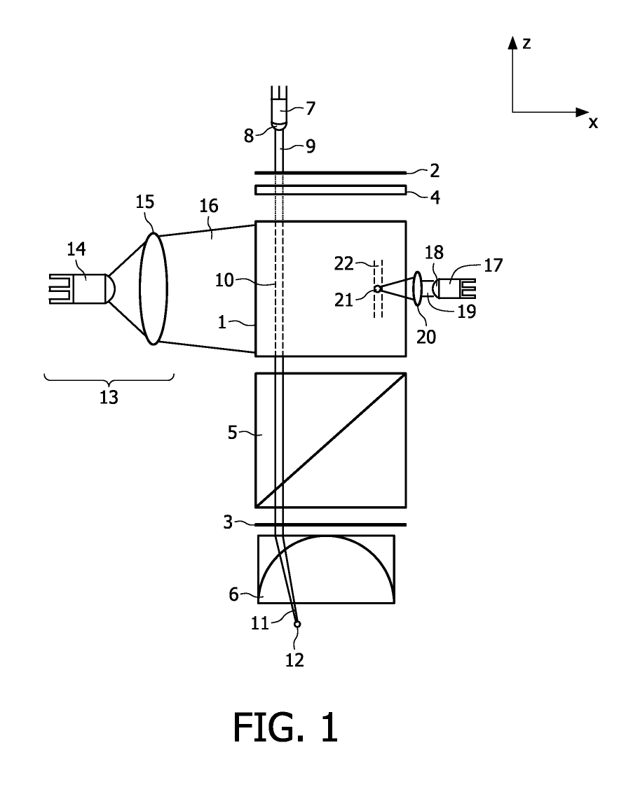

FIG. 1 very generally depicts a device that shows various inventive features.

Herein, 1 denotes a piece of laser material, in a laser cavity delimited by a first cavity mirror 2 and a second cavity mirror 3. A Q-switch is indicated by 4, and an optional polarizing beam splitter with 5. A focusing lens 6 is also optional.

A first diode 7 with a diode lens 8 emits a beam 9, which forms a laser beam 10 in the laser material 1, that is shaped to a focused beam 11 having a focal point 12.

An optical pump is indicated by 13, with a pump diode 14 and a lens 15, and emits a pump beam 16.

A diode 17 with diode lens 18, emits a beam 19 that is focused on a diode beam focal spot 21, causing a laser beam 22.

The laser material may be any material suitable for laser action with a desired wavelength. Such wavelength may e.g. be anywhere in the optical range, i.e. uv, visible or infrared. For shaving purposes, desirable wavelengths are around 800-2000, especially between about 1000 and 1100 nm. The laser material may be in the form of a rod, such as a crystal, or also as a container with a gas or fluid. An example of a useful laser material could be YAG, emitting at 1064 nm.

The cavity mirrors 2 and 3 may be any suitable laser mirrors, with a shape that fits the beam profile.

The Q-switch 4 may e.g. be a saturable absorber, such as Cr4+:YAG, for the above mentioned YAG-laser. Such a saturable absorber absorbs a large part of incident radiation, until a threshold is reached, at which point the absorption decreases to practically zero, and laser action can start.

The polarizing beam splitter 5 is an optional part, that serves a special purpose especially when using the device for detection, to be discussed further below.

Optical pump 13 comprises a diode 14, which emits sufficient energy, absorbable by the laser material 1, to bring the latter into simmer mode. Thereto, the diode 14 is optionally provided with collimating optics 15, and emits a pump beam 16. The wavelength and energy of the pump beam depend on the absorption characteristics and simmer threshold of the laser material 1, but may be readily selected by the skilled person, if necessary after some experiments. Alternatives for the diode 14 could be a pumping laser, a lamp, etc. Note that the “optical pump” relates to pump radiation of any suitable wavelength, and not particularly to laser wavelengths.

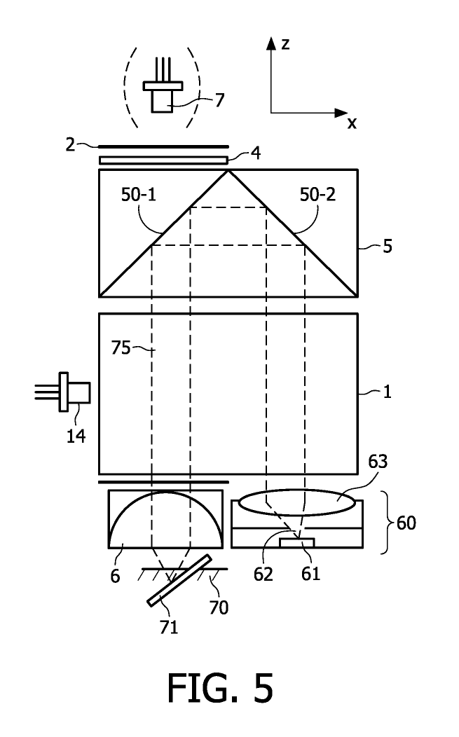

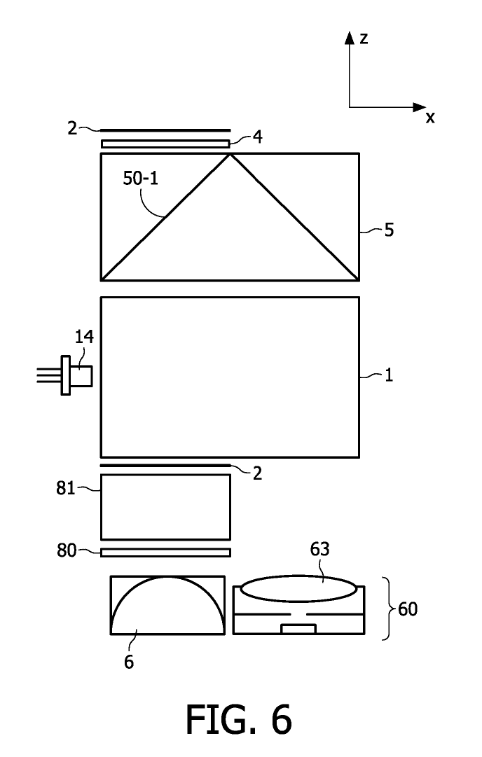

An important remark here is that the laser material 1 emits, in the pumped simmer state, a weak cw beam, though not a true laser beam. In most known laser devices that have a Q-switch, said switch is positioned at an output end of the device, to prevent low power “noise”. However, the present invention puts this low power cw radiation to good use, in that it serves as an illumination means that is emitted across the complete output surface of the laser material (or laser device) if a cavity mirror, filter, housing or the like would add extra constraints. Thereto, the Q-switch is positioned opposite the intended output face. Use of the simmer mode cw radiation will be elucidated in connection with FIGS. 5 and 6.

Diode 7, with a diode lens 8, is arranged to emit a substantially parallel, or at least narrow, beam 9 of radiation that is absorbable by the laser material 1. When such beam 9 is absorbed, a corresponding volume in the laser material will be excited above the laser threshold, and will show ASE (Amplified Spontaneous Emission), which, in the dashed volume, will saturate the absorber in its transmissive state, and start laser action in a localized laser action. In order to determine where the localized laser action is to be obtained, the diode 7 may be displaceable, or its beam 9 could be manipulated to be incident on a desired spot. This will be elucidated further below.

As an alternative, diode 17 with diode lens 18 could emit a beam 19 that is focused by lens 20 on a focal spot 21. The focal spot 21, and possibly a surrounding part of the volume of the laser material 1, may be excited above laser threshold, in a similar way to that described above, and provide a laser beam in the volume partly indicated with the short dashes. Thus, illumination from the sides is also possible. Even an unfocussed beam could provide laser action, when its intensity is high enough.

The laser beam 10 thus produced may be emitted at the second cavity mirror 3, and may be put to good use, e.g. illuminating or ablating an object. The lens 6 is optional, and could e.g. serve to provide a focused, high-intensity laser beam, for increased and more localized laser action, such as for precision operations. An example could be the generation of a laser-induced optical breakdown phenomenon (LIOB), as already discussed in the introductory part.

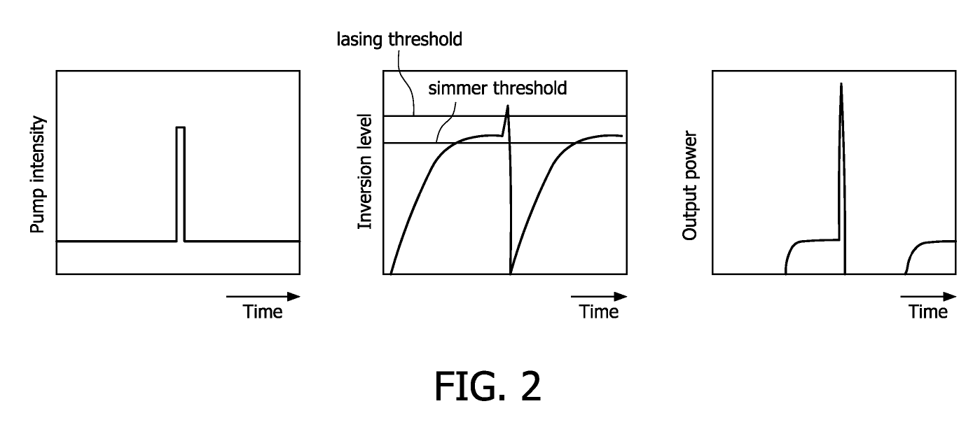

FIG. 2 diagrammatically shows a number of steps in the method of the invention, for three different quantities, from left to right the total pump intensity, i.e. radiation incident on the laser material, the inversion level (of relevant energy levels) and the output power. The three diagrams each show a distinct peak, which peaks substantially coincide in time.

The pump intensity reflects the more or less constant pumping, e.g. with the optical pump 13 of FIG. 1. The basic intensity is often rather low. The effect of this constant pumping is that it brings the molecules or the like of the laser material into a simmer mode, with an increased inversion level, but not yet with such an inversion level that laser action occurs. In the diagram in the middle, this is indicated by an inversion level that increases, under the influence of the pumping, to above the simmer threshold but below the laser threshold. The inversion level at the time of the pump intensity peak rapidly increases to above the laser threshold, and laser action suddenly sets in, causing a laser pulse to be emitted. Then also, the inversion level drops to zero, due to the stimulated emission. After that, the continuing pump intensity re-increases the inversion level to between the inversion and the laser threshold.

The resulting output power is shown in the diagram to the right. At first, there is no, or hardly any output. Then, when the simmer threshold is reached, a continuous wave output is obtained, indicated by the low level plateau. At the time of the pump intensity peak, the laser pulse is generated at a very much higher output level. After the laser pulse, the zero inversion level causes zero (or low) output, and the cycle can begin again.

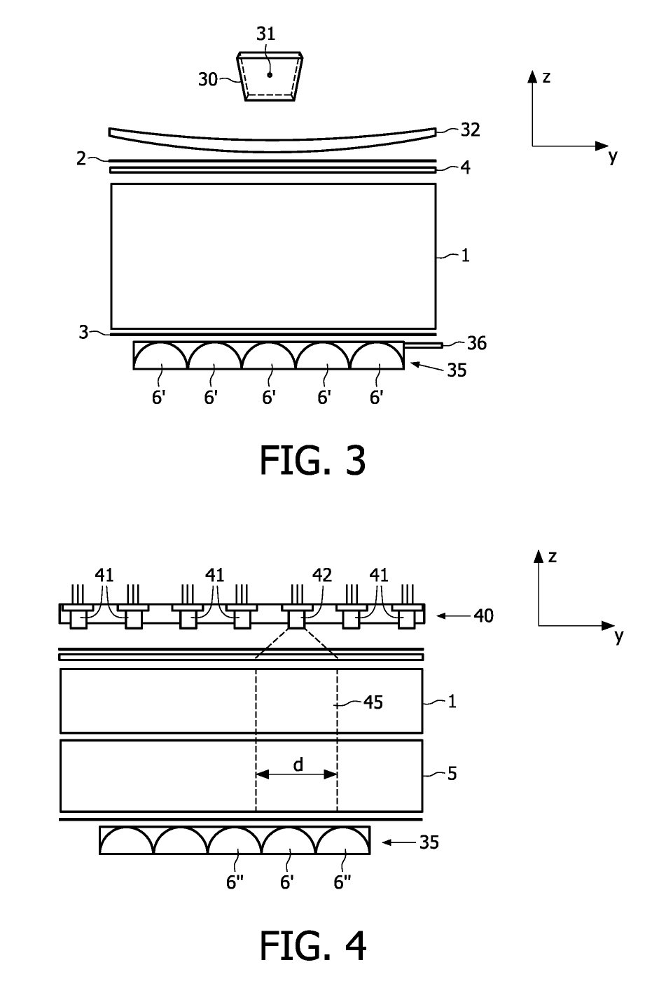

FIG. 3 diagrammatically shows a cross-sectional view of a device according to the invention. Herein, as in all Figures, similar parts are denoted by the same reference numerals.

The laser device still comprises a laser material 1 and Q-switch 4 between cavity mirrors 2 and 3. 30 denotes a beam deflection mirror, with a pivot point 31, and 32 is a scanning lens. An optical pump is not shown here.

A lens array 35, with focus lenses 6′ is movable with an array mover 36.

The beam deflection mirror 30 serves to aim an incident beam (not shown) at the laser material 1, and is pivotable about a pivot 31. In order to provide a beam that is incident in parallel, a scanning lens is provided. Preferably, the pivot 31 is in the focal point of the scanning lens 32. With this arrangement, a single source can provide a single beam, that can still address every part of the laser material 1.

In the device shown here, the lens array 35 is optional, and could be used to provide a focal spot, such as for LIOB to cut a hair or the like. Although a single movable lens could suffice, a multitude of lenses improves the speed of addressing, and also allows the provision of more than one laser beam pulse at the same time. Another very important advantage is that a large number of small lenses can much more easily provide an optical system with a high numerical aperture (NA). Such a high NA is safer when focused laser beams are used, since then the focal spot is limited in the beam direction. The field of view, both when detecting and when supplying pulses, is limited when compared to the size of the lens. However, when using a movable lens, and preferably an array of movable lenses, this may be compensated.

In practice, an NA of at least about 0.3, preferably at least about 0.6 is suitable for performing safe LIOB based cutting of hair in (human) skin tissue. For other purposes, some other NA could be expedient.

Furthermore, the lens array 35 can be moved into a corresponding correct position to guide the generated beam further, by means of the array mover 36, which may be any mechanical device, such as a piezo-electrical or electromechanical actuator, micro-motor and so on. The lens array 35 may e.g. be moved in the y-direction or in any other way in a plane perpendicular to the z-direction. The control of the array mover 36, by a control unit not shown here, may be coupled to the control of the beam deflection mirror. The positioning of the lens array 35 may be done in such a way that the focus lens 6′ that is closest to the desired position of the laser beam pulse is moved into position.

FIG. 4 diagrammatically shows a cross-sectional view of another device according to the invention.

Herein, 40 is a diode array, with diodes 41, 42. A beam splitter is denoted by 5. The diodes 41, 42 are individually addressable, and may each provide a beam 45, as does diode 42 in this case. The diode array 40 could also be made movable, in order to make the addressing of the laser material even more accurate and versatile. The diode array may be used to generate laser beam pulse patterns according to the way in which the diodes are addressed. Many uses are conceivable, e.g. in materials processing and so on.

The beam 45 generates a laser beam pulse of width d, which, however, need not be equal to the width of diode beam 45. In FIG. 4, the emitted laser beam pulse is addressing focus lens 6′, while neighboring focus lenses 6″ also are illuminated partly. In principle, this would lead to three focal points. In some cases, such as addressing individual optical fibers, this is not desirable. However, especially in the case of using the LIOB phenomenon, such as in cutting hairs or treating skin in a similar fashion, the fact that the focus lenses 6″ are not completely “filled” means that the total intensity in these additional focal points is (much) less than in the main focal point associated with lens 6′. Hence, LIOB will not occur and no damage need be done. In such case, the width d of the laser pulse beam may be larger than the diameter of the lenses 6′, 6″. Alternatively, the addressing of the lenses 6′, 6″ with respect to the beam need not be extremely accurate. Furthermore, in any practical situation, there will be inter-lens spaces, which then also form inherent safety zones. Suitable inter-lens dead spaces may depend on e.g. the number of lenses and diodes (or other light sources), the lens diameter and on the required safety level. For example, the lens dead space could be taken equal to the radius of the lens times the ratio of the number of lenses and the number of diodes. In the case of as many diodes as there are lenses, the required dead space could be (at least) one lens radius. Similar calculations may be made for all situations.

Also shown is the beam splitter 5, without any additional features. Such a beam splitter may be used for detection purposes, as will be explained below in connection with FIGS. 5 and 6.

FIG. 5 diagrammatically shows a detection device according to the invention.

It comprises a polarizing beam splitter 5, with, in this case two, beam splitter surfaces 50-1 and 50-2. Furthermore, a detector system 60 comprises a silicon photodetector 61 behind a pin hole 62 and a lens 63. Radiation returning from a hair 71 in skin 70 is formed into a beam 75.

In the detection device, with an optional addressing diode 7, the pump diode 14 is used to bring the laser material into simmer mode, in which it emits cw radiation. This radiation can enter the skin 70, preferably having a first polarization state. This radiation is reflected and scattered by various parts of the skin, not in the least by its surface, but also by a hair 71. The radiation from this hair returns with a probability that is polarization dependent. In any case, independent of polarization, some radiation will return through the focus lens 6 to the laser material 1. Since the laser material 1 is in simmer mode, it will amplify the incoming radiation, in beam 75. This beam is reflected at least partly at interfaces 50-1 and 50-2, again passes the laser material 1, by which it is amplified again. Note that the detector device could also be provided to the right of the laser material, e.g. when only a single interface 50-1 would be provided. Alternatively, more intricate patterns of interfaces 50-1, 50-2, . . . and/or additional mirrors could be provided, to create a path with even more amplification.

In any case, the amplified radiation 75 then passes the lens 63, the pin hole 62 and is incident on the photodetector 61. Since a pin hole 62 rejects a large fraction of the background radiation, and the returning radiation is amplified; the signal-to-noise ratio is improved. This may be further improved if use is made of a desirable state of polarization of the radiation incident on the skin, as has been described in European Patent Application EP 06125915.6, and which will be elucidated further in connection with FIG. 6. The detection device thus obtained shows a good ability to detect hairs, or other structures buried in materials such as skin, up to a relatively large depth, and with good sensitivity.

Note that a bias will be measured by the photodetector 61, due to the cw radiation from the simmer mode. However, it will be easy to subtract this bias from the measured signal. In fact, this bias may be put to good use. The device may generally be arranged to control the optical pump, such as pump diode 14, with the bias signal from the photodetector, such that the simmer mode is kept constant, e.g. with respect to the inversion level. That increases the accuracy of the measurements.

When the device further comprises an addressing (or pulse) diode 7, the device will be a laser treatment device, and may also be used to provide a laser pulse at a desired position, e.g. in order to cut the hair, with LIOB or the like. Thereto, once a hair position has been detected, a laser pulse may be generated by having diode 7 supply additional energy to bring the laser material 1 from simmer mode into laser mode, at least at the desired location. This desired location may be determined and addressed by means of the detected hair position. Then, the laser pulse reaches the detected hair position, and cuts it. In order to protect the detector against reflected parts of the laser pulse, a shutter may be built in, that shuts the detector system 60 when a laser pulse is generated, e.g. by closing the pin hole 62.

The device of FIG. 5 may be moved across the skin for shaving. Then, the device may detect hairs. The focus lens 6 may be movable, but could also be an array of lenses, which could be moved as well, e.g. reciprocatingly or rotatingly, in order to scan the skin while the device is moved.

Instead of the addressing diode 7, movable or not, the device could also e.g. comprise a set-up similar to FIG. 3, with a movable mirror 30 and a scanning lens 32.

FIG. 6 diagrammatically shows a cross-sectional view of an alternative embodiment of the detection device shown in FIG. 5.

This embodiment comprises two additional parts, viz. a quarter wave plate 80 and a Faraday rotator 81.

The Faraday rotator rotates the direction of the linearly polarized light over 45° clockwise or counter clockwise depending on the direction of travel through the rotator. The quarter wave plate 80 converts the linear polarization as output by the Faraday rotator to circularly polarized radiation, and vice versa. As described in EP 06125915.6, this is useful to further increase the sensitivity for radiation returning from subcutaneous hair. In this way the cross-polarized backscattered light remitted by the tissue will, after passing through the wave-plate, effectively be polarized in parallel with the incident light. By passing through the Faraday rotator in the reverse direction, the light will effectively be cross polarized compared to the light emanating from the laser cavity and, hence, will be separated by the polarizing beam splitter 5 to be detected by detection system 60. In any case, this detection device shows a very good sensitivity, as it is arranged to emit and detect circularly polarized light, and comprises the detection device according to the invention, i.e. comprises a light amplifier using a laser material in simmer mode.

If the device comprises a radiation source for additionally exciting the laser material 1 above laser threshold, the device of FIG. 6 could also be a laser treatment device, similar to the one described in connection with FIG. 5. Again, the laser treatment device could be arranged to provide a laser pulse at the position where a hair has been detected.

The devices shown in FIGS. 3-6 may be used for detecting and/or cutting hairs subcutaneously and so on, and then may be called a detection device and/or laser treatment device, in particular a hair detection and/or treatment device. Advantageously, these devices then comprise a hair recognition device, that is arranged to detect and recognize a hair from an image made by the detection device. For example suitable software or the like, in a control unit comprised in the device, to recognize a hair is known in the art. Furthermore, the laser treatment device for cutting hairs could include such a detection feature. The device could also cut hairs based on LIOB. Thereto, a pulse energy control device could be included, such as a control device arranged to control the size of the volume of laser material involved in the emitted laser pulse, as may be done by addressing a suitable number of pulse diodes, or across a suitable area of the laser material.

The present invention has been described with reference to a number of exemplary embodiments. The scope should, however, not be limited thereto, but should rather be determined by means of the appended claims.

1. A method of generating a laser beam, comprising
providing a laser device, having a laser material (1) with a surface from which a laser beam is emittable, and a Q-switch (4) in a laser cavity between cavity mirrors (2, 3);
bringing the laser material (1) into a simmer mode by supplying energy to the laser material (1);
supplying additional laser material absorbable energy to the laser material (1), such that in only a desired part of the laser material (1) a lasing threshold is exceeded, and a laser beam (10) is emitted from only a desired part of said surface.

2. A laser device, comprising
a laser cavity with cavity mirrors (2, 3), and with a laser material (1) having a surface from which a laser beam is emittable and a Q-switch (4) positioned in the laser cavity;
a pump means (14) able and arranged to supply energy (16) to the laser material (1) to bring the laser material (1) into a simmer mode;
a controllable source (7, 17; 41, 42) of additional energy arranged to supply laser material absorbable energy to the laser material (1), such that in only a desired part of the laser material (1) a lasing threshold is exceeded, and a laser beam (10) is emitted from only a desired part of said surface.

3. The laser device of claim 2, wherein the Q-switch (4) is positioned at a side of the laser material (1) opposite said surface of the laser material (1) from which the laser beam (10) is to be emitted.

4. The laser device of claim 2, wherein the source of additional energy comprises one or more diodes (7, 17; 41, 42), a pulsed laser or a flash lamp, and/or is arranged to supply the additional energy in a substantially parallel beam (9) or in a focused beam (21).

5. The laser device of claim 2, wherein the source (7, 17; 41, 42) of additional energy is positioned at the sides of the laser material (1) or at a back surface of the laser material (1) with respect to the emitted laser beam (10).

6. The laser device of claim 2, wherein the source (7, 17; 41, 42) of additional energy is controllable in that it comprises a plurality of independently addressable radiation sources (41, 42), or one or more radiation sources with separate shutters, or a radiation source and a controllable beam guiding device (30, 32).

7. A hair-removing device, comprising:
a laser source arranged to provide laser beam pulses;
an optical system (6; 6′; 6″) for focusing the laser beam pulses on a focal spot (12) and for positioning the focal spot (12) in a target position;
wherein the laser source comprises a laser device according to claim 2, and preferably wherein the optical system comprises a movable lens (6; 6′) or a plurality of individually addressable lenses (6, 6″).

8. The hair-removing device of claim 7, wherein the laser source is arranged to provide laser beam pulses with a predetermined pulse time, and wherein a dimension of the focal spot (12) and a power of the generated laser beam pulse are such that, in the focal spot, the laser beam pulse has a power density that is above laser induced optical breakdown (LIOB) threshold for hair tissue (71) for that predetermined pulse time.

9. The hair-removing device of claim 7, wherein the optical system (6; 6′, 6″) has a numerical aperture of at least 0.2, preferably at least 0.4, more preferably at least 0.6.

10. The hair-removing device of claim 7, comprising a plurality of movable lenses (6′, 6″), preferably arranged as a lens array.

11. The hair-removing device of claim 7, comprising a lens mover, in particular an array mover (36), that is arranged to impart an oscillating, reciprocating or vibrating movement to the movable lens (6; 6′, 6″), in particular to the lens array.

12. The hair-removing device of claim 7, comprising a lens mover or array mover, that is arranged to impart a rotating movement to the movable lens or lens array, respectively.

13. The hair-removing device of claim 7, further comprising an optical detection device, particularly to image subcutaneous parts of human skin (70), more particularly hairs (71), wherein the detection device comprises a photodetector (60) arranged to detect radiation that returns from an object to be detected.

14. The hair-removing device of claim 13, wherein the photodetector (60) is arranged and positioned to detect returning radiation that has passed through the laser material (1) of the laser device.

15. The hair-removing device of claim 13, wherein the photodetector comprises a photodiode, and/or a pin hole (62), that serves to select desired returning radiation and filter out unwanted background radiation, and/or an imaging lens (63) that images the returning radiation onto the photodetector.

16. The hair-removing device of claim 14, comprising a mirror device (50-1, 50-2) to guide the reflected radiation to the photodetector (60), in particular more than once through the laser material (1) of the laser device.

17. The hair-removing device of claim 16, wherein the mirror device comprises at least one polarizing beam splitter (50-1, 50-2).

18. The hair-removing device of claim 13, further comprising a hair recognition device.

19. The hair-removing device of claim 13, wherein the optical detection device comprises a shutter that is positionable in front of the photodetector (60).
